(19) United States
(12) Patent Application Publication
(10) Pub. No.: US 2020/0087403 A1
(43) Pub. Date: Mar. 19, 2020

(54) PHARMACEUTICAL COMPOSITIONS FOR THE TREATMENT OF THROMBOSIS IN PATIENTS SUFFERING FROM A MYELOPROLIFERATIVE NEOPLASM

(71) Applicants: INSERM (INSTITUT NATIONAL DE LA SANTÉ ET DE LA RECHERCHE MÉDICALE), Paris (FR); UNIVERSITÉ DE BORDEAUX, BORDEAUX (FR); CHU HÔPITAUX DE BORDEAUX, Bordeaux (FR); UNIVERSITÉ PARIS DIDEROT - PARIS 7, Paris (FR); UNIVERSITÉ PARIS XIII PARIS-NORD, Villetaneuse (FR)

(72) Inventors: Chloé JAMES, Pessac (FR); Thierry COUFFINHAL, Pessac (FR); Virginie GOURDOU-LATYSZENOK, Pessac (FR); Alexandre GUY, Pessac (FR); Martine JANDROT-PERRUS, Paris (FR)

(21) Appl. No.: 16/493,901

(22) PCT Filed: Mar. 14, 2018
(86) PCT No.: PCT/EP2018/056333

§ 371 Date: Sep. 13, 2019

(30) Foreign Application Priority Data
Mar. 15, 2017 (EP) 17305279.6

(51) Int. Cl. C07K 16/28; C12N 15/113; A61K 31/17; A61P 7/02

(52) U.S. Cl. CPC C07K 16/2854; C12N 15/1138; A61K 2039/505; A61P 7/02; C07K 2317/76; A61K 31/17

(57) ABSTRACT

Thrombosis is the main cause of morbidity and mortality in patients with JAK2V617F positive myeloproliferative neo-plasms (MPN). Recent works reported the presence of JAK2V617F in endothelial cells in some MPN patients. Here, the inventors show that JAK2V617F endothelial cells promote thrombosis through induction of endothelial P-selectin expression and thus demonstrate that P-selectin blockade was sufficient to reduce the increased propensity of thrombosis. Accordingly the present invention relates to a method of treating thrombosis in a patient suffering from a myeloproliferative neoplasm comprising administering to the patient a therapeutically effective amount of a P-selectin antagonist.

### FIELD OF THE INVENTION

The present invention relates to methods and pharmaceutical compositions for the treatment of thrombosis in patients suffering from a myeloproliferative neoplasm.

### BACKGROUND OF THE INVENTION

Myeloproliferative neoplasms (MPNs) are acquired clonal hematopoietic stem cell disorders, characterized by an increase in one or more myeloid lineages. The Philadelphia chromosome-negative (Ph−) MPNs include polycythemia vera (PV) with an excess of red blood cells, essential thrombocythemia (ET) with an increase of platelets and primary myelofibrosis (PMF) (Vardiman et al. 2002). More than 90% of patients with PV and half of those with ET and PMF carry a mutation in the Janus kinase 2 (JAK2) gene, ie. JAK2V617F (James et al. 2005, Baxter et al. 2005, Kralovics et al. 2005, Levine et al. 2005). JAK2 is a tyrosine kinase that initiates intracellular signaling of various type 1 cytokine receptors, such as erythropoietin and thrombopoietin receptors (Oh et al. 2010). The JAK2V617F mutation is responsible for a constitutive activation of the JAK2 kinase, resulting in subsequent activation of its downstream signaling pathways, ultimately leading to overproduction of myeloid cells.

Arterial and venous thromboses are the main causes of morbidity in Ph-negative MPNs with reported incidences ranging from 12-39% in PV and 11-25% in ET (Tefferi et al. 2012). The pathogenesis of thrombosis in MPN patients is complex and still largely elusive. It is clear that the hematopoietic system is involved in this pathophysiology: (i) Platelets isolated from MPN patients show signs of enhanced in vivo activation with increased P-Selectin expression (Falanga et al. 2005). (ii) Leukocytes were found to be activated in patients and hyperleukocytosis is an independent risk factor for thrombosis in PV, ET and PMF (Falanga et al. 2000, Falanga et al. 2005; Falanga et al. 2008, Falanga et al. 2007, Barbui et al. 2009, Alvarez-Arran et al. 2008). (iii) Red blood cells from PV patients also display increased adhesion to normal endothelium (Wautier et al. 2013). Several lines of evidence also argue in favor of endothelial cell (EC) activation in MPN patients: high levels of circulating endothelial cells (Belotti et al. 2012, Alonci et al. 2008) and increased endothelial activation markers such as soluble thrombomodulin, selectins and von Willebrand factor (Cella et al. 2010). Besides, 3 independent groups reported the presence of JAK2V617F not only in blood cells but also in endothelial cells from JAK2V617F positive MPN patients. Two independent studies used microdissection and revealed the presence of JAK2V617F in hepatic endothelial cells from JAK2V617F Budd Chiari Syndrome patients (Sozer et al. 2009) and in splenic endothelial cells from JAK2V617F myelofibrosis patients (Rosti et al. 2013). Another group detected the presence of JAK2V617F in circulating endothelial progenitor cells (ECFC) from 5/22 MPN patients (Teofili et al. 2011) and interestingly, all patients harboring JAK2V617F in ECFC were patients with a history of thrombosis, suggesting a strong association between the presence of JAK2V617F endothelial cells and the occurrence of thrombosis. Lastly, they observed that JAK2V617F-mutated endothelial progenitor cells from 4 patients displayed increased adherence to normal mononuclear cells (Teofili et al, 2011), suggesting that JAK2V617F endothelial cells could play an important role in the pathogenesis of thrombosis in MPN.

Under physiological conditions, endothelium maintains a hemostatic balance between both pro and antithrombotic factors. When stimulated by extrinsic factors such as inflammatory cytokines, hypoxia or antiphospholipid antibodies, endothelial cells become activated and promote thrombosis. Whether EC can become prothrombotic due to intrinsic modifications—such as genetic mutations—has never been demonstrated yet.

### SUMMARY OF THE INVENTION

The present invention relates to methods and pharmaceutical compositions for the treatment of thrombosis in patients suffering from a myeloproliferative neoplasm. In particular, the present invention is defined by the claims.

### DETAILED DESCRIPTION OF THE INVENTION

Thrombosis is the main cause of morbidity and mortality in patients with JAK2V617F positive myeloproliferative neoplasms (MPN). Today, key questions remain regarding the mechanisms involved in the development of thrombosis in these patients. Recent works reported the presence of JAK2V617F in endothelial cells in some MPN patients. Here, the inventors generated transgenic mice with inducible endothelial specific expression of JAK2V617F and determined that JAK2V617F endothelial cells are responsible for increased thrombus formation. Leukocytes were more adhesive to JAK2V617F endothelial cells, due to overexpression of membrane P-selectin, secondary to degranulation of Weibel-Palade bodies. P-selectin blockade was sufficient to reduce the increased propensity of thrombosis. Moreover, treatment with hydroxyurea also reduced thrombosis in these mice and the inventors demonstrated that hydroxyurea decreased the pathological interaction between leukocytes and JAK2V617F endothelial cells through direct reduction of endothelial P-selectin expression. Taken together, theses findings indicate that JAK2V617F endothelial cells promote thrombosis through induction of endothelial P-selectin expression. Besides, it provides the proof of concept that an acquired genetic mutation can make endothelial cells pro-thrombotic. This suggests that other activating mutations in endothelial cells could be causal in thrombotic disorders of unknown cause that account for 50% of recurrent venous thrombosis.

Accordingly, the present invention relates to a method of treating thrombosis in a patient suffering from a myeloproliferative neoplasm comprising administering to the patient a therapeutically effective amount of a P-selectin antagonist.

MPNs typically include polycythemia vera (PV), essential thrombocythemia (ET) and primary myelofibrosis (PMF). They are a diverse but inter-related group of clonal disorders of pluripotent hematopoietic stem cells that share a range of biological, pathological and clinical features including the relative overproduction of one or more cell types from myeloid origin with growth factor independency/hypersensitivity, marrow hypercellularity, extramedullary hematopoiesis, spleno- and hepatomegaly, and thrombotic and/or hemorrhagic diathesis. An international working group for myeloproliferative neoplasms research and treatment (IWG-MRT) has been established to delineate and define these conditions (see for instance Vannucchi et al, CA Cancer J. Clin., 2009, 59:171-191), and those disease definitions are to be applied for purposes of this specification.

In some embodiments, the patient harbours one mutation in JAK2. As used herein the term “JAK2” has its general meaning in the art and refers to the Janus Kinase 2 protein. The amino acid sequence of human JAK2 is well known in the art. Human JAK2 sequences are, for example, represented in the NCBI database (www.ncbi.orgwww.ncbi.nlm.nih.gov/), for example, under accession number NP_004963. Typical MPD associated mutation is the JAK2V617F mutation which refers to the point mutation (1849 G for T) in exon 14, which causes the substitution of phenylalanine for valine at codon 617 in the JAK homology JH2 domain. Other examples of JAK2 mutations include exon 12 mutations which can be substitutions, deletions, insertions and duplications, and all occur within a 44 nucleotide region in the JAK2 gene which encompasses amino acids 533-547 at the protein level. The most commonly reported mutations are small in-frame deletions of 3-12 nucleotides with a six nucleotide deletion being the most frequent. Complex mutations are present in one-third of cases with some mutations occurring outside this hotspot region. The N542-E543del is the most common mutation (23-30%), the K537L, E543-D544del and F537-K39delinsL represent 10-14%, and R541-E543delinsK comprise less than 10% of these mutations. JAK2 exon 12 mutations are located in a region close to the pseudo-kinase domain which acts as a linker between this domain and the Src homology 2 domain of JAK2.

As used herein, the term “thrombosis” has its general meaning in the art and is the process by which an unwanted blood clot forms in a blood vessel. It can occur in a vein or in an artery. Arterial thrombosis is the cause of almost all cases of myocardial infarction and the majority of strokes, collectively the most common cause of deaths in the developed world. Deep vein thrombosis and pulmonary embolism are referred to as venous thromboembolism, which is currently the third leading cause of cardiovascular-associated death. Thus the term “thrombosis” includes inter alia atrophic thrombosis, arterial thrombosis, cardiac thrombosis, coronary thrombosis, creeping thrombosis, mesenteric thrombosis, placental thrombosis, propagating thrombosis, traumatic thrombosis and venous thrombosis.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the P-selectin antagonist of the present invention is particularly suitable for the prophylactic treatment of thrombosis.

As used herein, the term “P-selectin” has its general meaning in the art and refers to a 140 kDa protein expressed by human platelets and endothelial cells, as described by Hsu-Lin et al, J Biol Chem 259: 9121 (1984), and Mc Ever et al, J Clin Invest 84:92 (1989). The term is also known as CD62P, GMP-140, PADGEM, and LECAM-3. This type I transmembrane glycoprotein (SwissProt sequence P16109) is composed of an NH2-terminal lectin domain, followed by an epidermal growth factor (EGF)-like domain and nine consensus repeat domains. It is anchored in the membrane by a single transmembrane domain and contains a small cytoplasmic tail.

As used herein, the term “P-selectin antagonist” includes any agent which is capable of antagonizing P-selectin, e.g., by inhibiting interaction between P-selectin and a P-selectin glycoprotein ligand-1, e.g., by inhibiting interactions of P-selectin expressing endothelial cells and activated platelets with PSGL-1 expressing leukocytes.

In some embodiments, the P-selectin antagonist is an antibody against P-selectin. The terms “antibody against P-selectin” and “anti-P-selectin antibody” refer to an antibody that is capable of binding to P-selectin with sufficient affinity such that the antibody is useful as a therapeutic agent in targeting P-selectin. The term “binding to P-selectin” as used herein means the binding of the antibody to P-selectin in either a BIAcore assay (Pharmacia Biosensor AB, Uppsala, Sweden) or in an ELISA in which either purified P-selectin or P-selectin CHO transfectants are coated onto microtiter plates.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments (e.g. Fab, Fab′, F(ab′)2 or scFv . . . ). In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. polypeptides of the present invention). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

In some embodiments, the monoclonal antibody of the invention is a chimeric antibody, in particular a chimeric mouse/human antibody. As used herein, the term “chimeric antibody” refers to an antibody which comprises a VH domain and a VL domain of a non-human antibody, and a CH domain and a CL domain of a human antibody.

In some embodiments, the monoclonal antibody of the invention is a humanized antibody. In particular, in said humanized antibody, the variable domain comprises human acceptor frameworks regions, and optionally human constant domain where present, and non-human donor CDRs, such as mouse CDRs. According to the invention, the term “humanized antibody” refers to an antibody having variable region framework and constant regions from a human antibody but retains the CDRs of a previous non-human antibody.

In some embodiments, the monoclonal antibody is a human monoclonal antibody. As used herein the term “human monoclonal antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human monoclonal antibody”, as used herein, is not intended to include antibodies in which CDR sequences derived from the germline of another mammalian species, such as a mouse, have been grafted onto human framework sequences.

In some embodiments, the monoclonal antibody of the present invention is selected from the group of Fab, F(ab′)2, Fab′ and scFv. As used herein, the term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond. The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin. The term “Fab′” refers to an antibody fragment having a molecular weight of about 50,000 and antigen binding activity, which is obtained by cutting a disulfide bond of the hinge region of the F(ab′)2. A single chain Fv (“scFv”) polypeptide is a covalently linked VH::VL heterodimer which is usually expressed from a gene fusion including VH and VL encoding genes linked by a peptide-encoding linker. The scFv fragment of the invention includes CDRs that are held in appropriate conformation, preferably by using gene recombination techniques.

The antibodies of the present invention are produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In some embodiments, the monoclonal antibody of the present invention does not mediate antibody-dependent cell-mediated cytotoxicity and thus does not comprise an Fc portion that induces antibody dependent cellular cytotoxicity (ADCC). In some embodiments, the monoclonal antibody of the present invention does not comprise an Fc domain capable of substantially binding to a FcγRIIIA (CD16) polypeptide. In some embodiments, the monoclonal antibody of the present invention lacks an Fc domain (e.g. lacks a CH2 and/or CH3 domain) or comprises an Fc domain of IgG2 or IgG4 isotype. In some embodiments, the monoclonal antibody of the present invention consists of or comprises a Fab, Fab′, Fab′-SH, F(ab′)2, Fv, a diabody, single-chain antibody fragment, or a multispecific antibody comprising multiple different antibody fragments. In some embodiments, the monoclonal antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by ldusogie et al.

Antibodies against P-Selectin are known from, e.g., U.S. Pat. No. 4,783,399, WO 93/06863, Geng et al (J. Biol. Chem., 266 (1991) 22313-22318), WO 93/21956, WO 2005/100402 and WO2008069999. In some embodiments, the anti-P-selectin of the present invention is SEG101 also named crizanlizumab.

In some embodiments, the P-selectin antagonist is hydroxycarbamide, also known as hydroxyurea.

In some embodiments, the P-selectin inhibitor is an inhibitor of P-selectin expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of P-selectin mRNA by binding thereto and thus preventing protein translation or increasing mRNA degradation, thus decreasing the level of P-selectin, and thus activity, in a cell. For example, antisense oligonucleotides of at least about 15 bases and complementary to unique regions of the mRNA transcript sequence encoding P-selectin can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. P-selectin gene expression can be reduced by contacting a patient or cell with a small double stranded RNA (dsRNA), or a vector or construct causing the production of a small double stranded RNA, such that P-selectin gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing P-selectin. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Ban viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1l) in Zetsche et al. (“Cpf1 is a Single RNA-guided Endonuclease of a Class 2 CRISPR-Cas System (2015); Cell; 163, 1-13).

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the antibody or antibody portion are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. An exemplary, non-limiting range for a therapeutically effective amount of an antagonist of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time.

Typically, the antagonist is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1 Increased endothelial P-selectin expression is responsible for the pro-adhesive phenotype of JAK2V617F endothelial cells. In static conditions, increased adhesion of normal mononuclear cells (a) and neutrophils (b) on JAK2V617F HUVECs is reversed in the presence of a P-selectin blocking antibody. In Pdgfb-iCreERT2;JAK2^{V617F/WT} mice, increased rolling (c) and adhesion (d) of leukocytes is abolished in the presence of a P-selectin blocking antibody. (e) Increased thrombus formation in Pdgfb-iCreERT2;JAK2^{V617F/WT} mice is abrogated in the presence of a P-Selectin blocking antibody. Results are mean value+/−s.e.m. Statistical significance by 2-way ANOVA analysis of variance and Sidak post-hoc test. **P<0.05; **P<0.01; ****P<0.0001.

### EXAMPLE

Materials. For Western-Blotting in human endothelial cells, the following antibodies were used: JAK2 (rabbit, Santa Cruz Biotechnology, Dallas, Tex.), JAK2 Tyr1007/1008 (rabbit, Cell Signaling, Danvers, Mass.) STAT-3 (mouse, Cell Signaling), STAT3 Tyr705 (rabbit, Cell Signaling), pan-Akt (9272, rabbit, Cell Signaling), Ser473 p-Akt (rabbit, Cell Signaling), α-Tubuline (rabbit, Cell Signaling). For P-Selectin immunostaining and blocking in mice, anti-mouse P-Selectin monoclonal antibody was used (RB40.34 clone, BD Biosciences, Franklin Lakes, N.J.). Blocking effect of this antibody was previously described^{39,40}. For vWF immunostaining in human endothelial cells, primary antibody rabbit anti-human vWF (EMD Millipore) was used. For VE-Cadherin staining in human endothelial cells, monoclonal antibody anti-VE-Cadherin (SantaCruz Biotechnology) was used.

Cell culture/Lentivirus transduction. Human umbilical venous endothelial cells (HUVECs, Lonza, Basel, Swiss) were cultured in EGM-2 media (Lonza, CC-3156) supplemented with EGM-2 Single Quots (Lonza, CC-4176). Cells were transduced with GFP lentivirus encoding human JAK2V617F or JAK2WT by adding lentiviral supernatant to the medium and incubating overnight at 37° C. An empty lentivirus encoding only for GFP was used as a negative control. Lentivirus were transduced at a multiplicity of infection of 20, allowing more than 95% of GFP positive cells. Transgene stability was verified after each passage and remaining stable throughout the passages. HUVECs were used for experiments between passage 5 and passage 7.

Western-Blot in HUVECs. HUVECs were platted in 6 wells plats to reach confluence. Confluent cells were then starved for 4 hours in EBM2 medium (0% SVF, 0% BSA). Cells were lysed in Laemmli buffer (Tris 10 mM, Saccharose 7%, SDS 2%, (β-mercapto-ethanol 3.92%, Blue of bromophenol 0.04 g/L) after congelation at −20° c. Cell lysates were resolved by SDS-PAGE and probed with following antibodies: JAK2 (rabbit, Santa Cruz Biotechnology), JAK2 Tyr1007/1008 (rabbit, Cell Signaling), STAT-3 (mouse, Cell Signaling), STAT3 Tyr705 (rabbit, Cell Signaling), pan-Akt (rabbit, Cell Signaling), Ser473 p-Akt (rabbit, Cell Signalling), α-Tubuline (mouse, Cell Signaling). Binding of antibodies to the blots was detected using Odyssey Infrared Imaging System (LI-COR Biosciences, Lincoln, Nebr.).

Thrombin generation. Thrombin generation was measured in freshly prepared platelet free plasma by means of the Calibrated Automated Thrombogram (CAT) method (Thrombinoscope BV, Maastricht, Netherlands). Thrombin generation was performed in 96-well plates containing confluent HUVECs washed with HEPES buffer (20 mM Hepes, 140 mM NaCl, 5 mg/ml BSA, pH 7.35). Polystyrene was considered as the control condition since it is the reference material to study thrombin generation⁴¹. Thrombin generation was triggered by tissue factor (1 or 2.5 pM final concentration). The velocity index (nM/min) was calculated as the ratio Peak/(time to Peak-LagTime)]; LagTime (LT) is related to the initiation phase of coagulation, time to Peak (ttPeak) and Peak are the reflection of the amplification phase of coagulation and the Endogenous Thrombin Potential (ETP) reflects the global quantity of thrombin produced during the experiment.

Protein C activation. Confluent monolayer HUVECs in 96-well plates were washed twice with warm sterile PBS and then incubated with thrombin (2 nM thrombin) and CaCl2 (2.5 mM) for 10 min at 37° C. Protein C was then added to each well at a final concentration of 0.2 μM. The plate was further incubated for different times at 37° C. Aliquots were then collected and transferred to a clean 96-well plate. Hirudin (100 U/ml) was added to block thrombin. Activated protein C activity was monitored during 30 minutes at 405 nm on a FLUOStar Optima plate reader (BMG Labtech GMBH, Ortenberg, Germany) using a specific substrate (PNAPEP™1566, 100 μL at 0.4 mM). Results were plotted as the rate of substrate hydrolysis as the function of time of PC activation.

In vitro static adhesion on endothelial cells. Blood samples were performed from well healthy voluntary witnesses. Blood was diluted with PBS (1/2 dilution) and deposed on lymphocyte separation medium (Pancoll, Dominique Dutscher, Brumath, France) before centrifugation at 450 g during 20 minutes to obtain mononuclear cells (MNC). Monocytes were isolated from MNC by magnetic immunoseparation and selection of CD14 positive cells (EasySep Monocyte Extraction kit, R&D Systems, Minneapolis, Minn.). Neutrophils were obtained from blood samples deposed on neutrophils separation medium (Polymorphprep, Fresenius Kabi, Oslo, Norway) before centrifugation at 450 g during 45 minutes. After isolation, MNCs, monocytes and neutrophils were marked with membrane dye (CellTracker Orange, ThermoFisher Scientific, Waltham, Mass.). Transduced JAK2V617F HUVECs, wild type HUVECs or negative control HUVECs were platted in 24 wells plats to reach confluence. Mononuclear cells, monocytes, and neutrophils were added on top of transduced HUVECS for 1 hour at 37° C., using 500 000 cells by well. After 1 hour, three washes with EGM2 medium were done. We visualized adherent cells using a fluorescent microscope (AxioObserver, Zeiss, Oberkochen, Germany) and analysed images by ZEN imaging software (Zeiss). Well surface was quantified in order to obtain the following ratio: number of adherent cells/mm2. For P-Selectin inhibition experiments, we used P-Selectin blocking antibody (AK4 clone, BioLegend, San Diego, Calif.) during 30 minutes before deposit of cells on HUVECs.

In vitro Neutrophils adhesion on HUVEC cells in flux conditions. Canals in a cell chamber (Vena8 Endothelial+, Celix No: 1510-02) were coated with Human fibronectin (100 ng/ml) (Promocell, Heidelberg, Germany, No: C-43060), before seeding of pre-cultivated HUVECs at the concentration of 3.10⁶ Cells/ml. After two hours at 37° C., cells were cultivated with flux in a closed circuit where a pump was linked to medium and linked to cells in canals (KIMA-IPOD TOUCH MICROFLUIDIC PUMP, Cellix). A flux with two periods was imposed to the cells: a perfusing period (3 minutes with 600 μL/min) and a rest period (20 minutes with 0 μL/min). Cells were incubated with this flux for 48 hours at 37° C. 3.10⁶ Neutrophils/ml were isolated from a healthy donor with a kit (MACSxpress, Neutrophil cocktail human-lyophilized, Miltenyi Biotec, Bergisch Gladbach, Germany, No: 130-104-434) before introduction in canals with a venous flux of 10 μL/min and observed at 20× in phase contrast with a microscope ZEISS during a film of 5 minutes. After five minutes canals were washed with medium and pictures of every canal were performed. Neutrophils quantification was performed blindly on canal's picture and Neutrophils Velocity is studied with FIJI (Image J).

Generation and characterization of Pdgfb-iCreERT2;JAK2^{V617F/WT} mice. The conditional flexed JAK2 (JAK2V617F/WT) mice were generously provided by J. L Villeval and have been previously described¹⁹. The double-heterozygous Pdgfb-iCreERT2;JAK2^{V617F/WT} mice were generated by crossing JAK2^{V617F/WT} mice with Pdgfb-iCreERT2 mice allowing tamoxifen-inducible adult expression of JAK2^{V617F} in endothelium. Littermate Pdgfb-iCreERT2-negative;JAK2^{V617F/WT} mice were used as controls. To induce Cre activity in Pdgfb-iCreERT2;JAK2^{V617F/WT} mice, we used oral gavage of a single dose of 8 mg of tamoxifen. Tamoxifen induction was performed in 5 weeks-old animals. Mutant mice were analysed 2-3 weeks after tamoxifen administration. Ears of adult mice were genotyped by PCR. Haematocrit, hemoglobin level, platelet, and white cells count were determined using an automated counter (scil Vet abc Plus+) on blood collected from the sublingual vein in EDTA containing tubes. For blood flow cytometry analysis in Pdgfb-iCreERT2;mT/mG;JAK2^{V617F/WT} mice, cells were stained with TER-119 APC (BD Biosciences, Ter-119 clone), CD42d-APC (BioLegend, 1C2 clone) and Ly6-APC (BD Biosciences, RB68C5 clone). FACS analysis was performed using an Accuri C6 flow cytometer (BD Biosciences). Data were interpreted using BD Accuri C6 Analysis Software.

Isolation of endothelial cells from mice. Mice were euthanized followed by exposure of the thoracic and abdominal cavity. In order to isolate ECs from lungs, after the right atrium was cut, physiologic sera was injected in the left ventricle to completely flush blood cells from the lungs. Kidneys and lungs were removed and minced into small pieces, following by incubation for 60 minutes at 37° C. with 5 ml 0.1% type 4 collagenase. The digested tissue suspension was aspirated into to a 10-ml syringe with a 14-gauge cannula, and clumps were triturated into a single-cell suspension. The single-cell suspension was filtered through a 70μm strainer. The filtered cell suspension was centrifuged for 10 minutes at 300 g, and the cell pellet was washed with 0.5% BSA, 2 mM EDTA, and PBS containing CaCl2 and MgCl2 (Gibco, ThermoFisher Scientific). The cell pellet was suspended with 190 μL 0.5% BSA, 2 mM EDTA, PBS, following by the addition of 5 μL anti-CD31 antibody (BD Biosciences, 553370) and incubation at 37° C. during 30 minutes. After washing in PBS-EDTA-BSA, 200 μL of anti-rat beads were added to the cell suspension, and cells were incubated 15 minutes at 4° C. Cell suspension was next washed in PBS-EDTA-BSA and endothelial cells were isolated using magnetic microbeads (Miltenyi Biotec, Bergisch Gladbach, Germany, 120-000-291).

DNA purification and quantitative allele-specific PCR. Genomic DNAs were purified using NucleoSpin® Tissue kit (Macherey-Nagel, Duren, Germany). For quantification of wild type (WT) and mutated JAK2 DNA, quantitative allele specific PCR from gDNA was conducted to identify amplified fragments from the mutated or WT JAK2 DNA, respectively. It was performed on a 7500 Real Time PCR System AB (Applied Biosystems, Foster City, USA) and analyzed with associated software.

Intravital microscopy of mesenteric venules. Pdgfb-iCreERT2;JAK2^{V617F/WT} mice and Pdgfb-iCreERT2negative;JAK2^{V617F/WT} mice were used 20 days post tamoxifen injection. Intra-peritoneal injection of TNF-alpha (R&D Systems) at the dose of 250 ng/mice was performed. 4 hours after TNF-alpha administration, mice were anesthetized with intra-peritoneal injection of ketamine/xylazine. Rhodamin 6G (Sigma-Aldrich, Saint-Louis, Mo., ref: 4127) injection was performed to stain leukocytes five minutes before incision. An incision was made through the abdominal wall to expose the mesentery, and mesenteric venules of 150- to 250-μm diameter were studied. 5 venules by mice were sequentially observed for 1 minute and 30 seconds during the 30 minutes after surgical procedure, using a fluorescent microscope (AXIO Zoom.V16, Zeiss). For P-Selectin inhibition, 25 μg/mouse of P-selectin blocking antibody (RB40 clone, BD Biosciences) or isotype control antibody (A110-1 clone, BD Biosciences) was injected 5 minutes before starting the analysis.

Video analysis. Rolling leukocytes were quantitated by counting the number of rhodamin-marked cells passing a given plane perpendicular to the vessel axis in 30 seconds. Adherent leukocytes were quantitated by counting the number of rhodamin-marked cells motionless during 30 seconds. Vessel surface was quantitated to perform the following ratio: adherent cells/vessel surface (number of cells/cm2). Videos analyses were performed using ZEN imaging software (Zeiss). Rolling and adhesion quantification were performed by two independent observers, blindly.

Mouse model of thrombus formation. 20 days post-tamoxifen induction, PDGFb-iCreERT2;JAK2V617F^{V617F/WT} mice and PDGFb-iCreERT2-negative;JAK2WT^{V617F/WT} mice were utilized for experiments. We studied spontaneous thrombus formation, and two thrombosis-induced models. To induce platelet and coagulation activation, intra-peritoneal injection of collagen (Nycomed Pharma, Zurich, Swiss, 7806141/450) at the dose of 75 μg/kg and epinephrine (Helena Laboratories, Beaumont, Tex., 5367) at the dose of 30 μg/kg was performed 3 minutes before euthanasia. To induce low inflammation, TNF-alpha injection (RD Systems, 210-TA-020) at the dose of 250 ng/mice was injected 4 hours before euthanasia. The dose of 500 ng/mice TNF-alpha is commonly used to trigger inflammation^{43,44} and we chose a lower dose to reveal a potential hypersensitivity. After injection of collagen-epinephrine or TNF-alpha, mice were anesthetized with isoflurane and blood was obtained by sub-lingual sampling in polypropylene Eppendorf tubes containing 5 μL of EDTA in order to perform blood count. After euthanasia, an incision was performed in thoracic wall to expose mice heart and lungs were washed with intra-cardiac perfusion of PBS without CaCl2 and MgCl2 (Gibco ThermoFisher Scientific) during 3 minutes. Lungs were fixed with secondary three-minutes injection of 10% formalin and collected before formalin fixation and paraffin embedding. For P-selectin inhibition, we used P-selectin blocking antibody (RB40 clone, BD Biosciences) at the dose of 25 μg per mouse, 4 hours before euthanasia.

Histology. To quantify thrombus formation in mice, Carstair's staining was performed. Slides were hydrated in xylol and ethanol to distilled water, followed by incubation in 5% ferric ammonium sulfate for 5 min, washing, and staining by Mayer hematoxylin for 5 min, washing, and Picric Acid-orange G solution for 1 hour, and washing, 1% phosphotungstic acid for 10 min. After washing, slides were stained by Ponceau Fuchsin solution for 7 min, washing, 1% phosphotungstic acid for 10 minutes, Anilin blue solution for 30 min, and rinsed in distilled water. Slides were dehydrated covered with a coverslip using mounting medium. Thrombus quantification was performed using an optical microscope and pulmonary area was quantitated to perform the following ratio: thrombi number/pulmonary area (number of thrombi/cm2). Three slides by mice were analysed by two independent observers, blindly.

Immunostaining. Immunofluorescence analyses were realized on HUVECs, with or without activation of cells by TNF-alpha (10 ng/ml overnight). Cells were fixed with 2% paraformaldehyde (PFA) for 10 minutes. After saturation in 5% bovine serum albumin for 1 hour, cells were incubated with primary antibody rabbit anti-human vWF (Merck Millipore), vWF primary antibody was resolved with Alexa Fluor 588 conjugated secondary antibody (Invitrogen, Carlsbad, Calif.). Cells were mounted in Vectashield mounting medium containing 4,6-diamidino-2-phenylindole (DAPI, Vector Laboratories, Burlingame, Calif.), imaged with a confocal microscope (Zeiss LSM 700) and analysed by Imaris software (Bitplane). In mice, P-Selectin immunostaining was performed in carotid arteries of PDGFb-iCreERT2;JAK2V617F^{V617F/WT} and control mice. Briefly, mice were euthanized followed by exposure of the thoracic cavity. After the right atrium was cut, PBS was injected in the left ventricle to completely flush blood cells from the carotid arteries. Fixation of endothelial cells was performed by injection of 10% formalin. After exposure, carotid arteries were removed from the mice and fixed in 10% formalin during 10 minutes, following by washing in PBS and saturation in 10% donkey serum-PBS. Arteries were incubated with primary anti P-Selectin antibody (RB40.34, BD Biosciences) and primary anti VE-Cadherin antibody (SantaCruz, sc-6458,) overnight at 4° C. After three washing with PBS, arteries were incubated with Alexa Fluor 568 conjugated secondary antibody (VE-Cadherin) or and Alexa Fluor 488 conjugated secondary antibody (P-Selectin) during 2 hours at room temperature. Arteries were then mounted in Vectashield mounting medium containing 4,6-diamidino-2-phenylindole (DAPI, Vector Laboratories), imaged with a confocal microscope (Zeiss LSM 700) and analyzed with Image J.

Quantification of VWF in HUVEC supernatant and in HUVECs. HUVEC cell were seeded in 12 wells plate (Costar). When they were confluent, they were washed with 500 μL of PBS (Gibco ThermoFisher Scientific) before adding 500 μL of medium not deprived. Experiments were performed in absence or in presence of TNF-alpha (10 ng/ml during 24 hours) (Merck Millipore). After 24 hours, supernatant was removed, centrifuged at 12000 g during 5 minutes and stored at −80° C. To quantify vWF in HUVECs, cells were washed with PBS before adding in wells 500 μL of PBS. Cells were destroyed in wells with scrapper. Liquid was removed and 5 freezing and thawing successions were performed. Finally, the intracellular lysates were centrifuged at 12000 g during 5 minutes and stored at −80° C. As previously published⁴⁵, vWF was quantified following this method: 98 wells plate (Greiner, Flat Bottom) were coated with an anti-vWF antibody (DAKO, Les Ulis, France, A0082) diluted at 1/660 in a coating buffer overnight at 4° C. After washing and blocking, sample of HUVEC supernatant and intracellular lysate were put in wells. Standards were realized with a platelet poor plasma from a healthy donor. After 2 hours and a second wash, antibody anti-vWF coupled to HRP (DAKO, P0226) diluted at 1/6000 in wash buffer was deposit in wells. After 2 hours and a third wash, HRP was revealed with an OPD and H2O2 solution. After 2 minutes of coloration, the reaction was blocked with a H2SO4 3M solution and reading was performed at 492 nm with OPTIMA plate reader.

Study of hydroxyurea effect on endothelial cells. HUVECs were treated with HU (Sigma-Aldrich, H8627) during 24 hours at the concentration of 100 μM before washing with EGM2 medium. Neutrophils static adhesion was performed as described. To study the effect of hydroxyurea in mice, Pdgfb-iCreERT2;JAK2^{V617F/WT} and control mice were treated with HU at the dose of 200 mg/kg/day (oral gavage) during 10 days before experiments.

ELISA for soluble P-Selectin. Mice were anesthetized, and blood was obtained by retro-orbital venous plexus sampling in polypropylene Eppendorf tubes containing 100 μL of ethylenediaminetetraacetic acid (EDTA). Plasma was prepared by centrifugation of the blood within 30 minutes at 1000 g for 10 min at +4° C. then 10 000 g for 15 min at +4° C. Enzyme-linked immunoabsorbent assay (ELISA) was performed according to manufacturer's instructions (R&D Systems).

Von Willebrand Factor quantification in mice. Mice were anesthetized, and blood was obtained by retro-orbital venous plexus sampling in polypropylene Eppendorf tubes containing 0.138 M sodium citrate (1/10 volume). Plasma was prepared by centrifugation of the blood 20 minutes à 1500 g. Plasma vWF concentration was measured by ELISA using a polyclonal antibody against vWF (Dako France, Les Ulis, France, ref A0082) and a horseradish peroxidase-conjugated secondary antibody anti VWF (Dako, ref P0226). Pooled plasma from 40 C56BL/6 WT mice was used as reference and set at 100%. Results were expressed as a percentage of the normal murine vWF level.

Statistics. Results were expressed as mean±SEM. Statistical significance was calculated using the Student t test or Mann-Whitney statistical test to compare differences between 2 groups. To compare difference among multiple groups, 1-way-ANOVA followed by Tukey post-hoc test or 2-way-ANOVA followed by Sidak post-hoc test were used. GraphPad Prism 6 was used. P value of less than 0.05 was considered significant.

Study approval. All mice used in this study were bred and maintained at the institute. This study was conducted in accordance with both Bordeaux University institutional committee guidelines (committee CEEA50) and those in force in the European community for experimental animal use (L358-86/609/EEC).

Data availability. The authors declare that the data supporting the findings of this study are available within the article and from the authors on reasonable request.

### Results

### The Expression of JAK2V617F by Endothelial Cells Leads to Increased Thrombus Formation

To analyze the specific role of JAK2V617F endothelial cells in thrombus formation, we crossed Pdgfb-iCreERT2 mice with conditional flexed JAK2 (JAK2V617F/WT) mice¹⁹, to allow heterozygous expression of JAK2V617F in endothelial cells but not in hematopoietic cells after tamoxifen administration. We then investigated whether Pdgfb-iCreERT2;JAK2^{V617F/WT} mice displayed a higher propensity for thrombosis. We looked at pulmonary thrombus formation using experimental conditions that allowed assessment of endothelial involvement, i.e. without exposition of the subendothelium. We thus used three conditions to strongly validate our observations: (i) spontaneous thrombosis, (ii) a mild thrombosis model consisting of the administration of low doses of collagen plus epinephrine, as we reasoned we would induce specifically a weak activation of platelets and vasoconstriction to better evidence an intrinsic prothrombotic phenotype of JAK2V617F endothelial cells, (iii) a weak inflammatory trigger of thrombosis with injection of low doses of TNF-alpha to reveal a potential hypersensitivity to inflammation. Small spontaneously formed thrombi were observed in the lungs of Pdgfb-iCreERT2;JAK2^{V617F/WT} mice, but not in littermate JAK2^{V617F/WT} control mice. With the 2 models of mild induction of thrombosis, we observed significantly increased thrombus formation in Pdgfb-iCreERT2;JAK2^{V617F/WT} mice compared with controls. Altogether, these results demonstrated that JAK2V617F endothelial cells have a prothrombotic phenotype.

### JAK2V617F Endothelial Cell Have Normal Anticoagulant Activity

We then wanted to decipher the mechanisms by which endothelial JAK2V617F expression leads to a prothrombotic phenotype. Healthy endothelial cells are capable of inhibiting coagulation and thus thrombin generation in several ways: secretion of Tissue Factor Pathway Inhibitor, expression of Thrombomodulin and Endothelial Protein C receptor. Indeed, in the presence of human umbilical vein endothelial cells (HUVEC), Tissue Factor (TF)-triggered thrombin generation in platelet poor plasma is considerably impaired²⁰. To assess whether the expression of JAK2V617F by endothelial cells decreased their anticoagulant properties or even triggered procoagulant properties, we measured thrombin generation at the surface of HUVECs transduced either with lentivirus encoding human JAK2V617F or JAK2 wild-type (JAK2WT) or empty lentivirus as controls. Western blot analysis revealed induced protein expression of JAK2 and an increase in the phosphorylation level of JAK2, STAT3 and AKT in JAK2V617F HUVECs, in agreement with an hyperactivation of the JAK/STAT pathway. Under resting conditions, measurement of thrombin generation at the surface of control, JAK2WT- or JAK2V617F-lentivirus infected endothelial cells did not reveal any significant difference in the kinetic and extent of thrombin generation. These results rule-out a significant gain of procoagulant activity in response to JAK2WT or JAK2V617F-induced endothelial expression. We reasoned that JAK2V617F endothelial cells could acquire a procoagulant phenotype due to the exposure to circulating inflammatory stimuli. We thus repeated the same experiments after overnight activation with TNF-alpha, but did not observe any difference. Additionally, we measured the rate of thrombin-triggered protein C activation and did not observe any difference between the cells demonstrating that JAK2V617F endothelial cells have normal anticoagulant properties.

### JAK2V617F Endothelial Cells Have a Pro-Adhesive Phenotype

Exposition of endothelial cells to inflammatory stimuli leads to endothelial expression of adhesion molecules that which allows the rolling and adhesion of leukocytes. Teofili et al. reported increased adhesion of normal human mononuclear cells (MNC) on patients' endothelial cells derived from JAK2V617F ECFC, in static conditions. We first assessed whether our model of JAK2V617F transduced HUVECs reproduced such a proadhesive phenotype. We indeed observed increased adhesion of healthy MNCs and polymorphonuclear neutrophils (PMN). We then addressed the adhesive properties of JAK2V617F HUVECs in flow conditions and reported that more normal PMNs rolled and stably adhered to JAK2V617F-HUVECs as compared to JAK2WT-HUVECs. To assess whether this proadhesive phenotype was also observed in vivo, we visualized leukocyte interactions with mesenteric venules from Pdgfb-iCreERT2;JAK2^{V617F/WT} mice. We found that both leukocyte rolling and adhesion were significantly increased in Pdgfb-iCreERT2;JAK2^{V617F/WT} mice only when they were previously administered with low dose TNF-alpha.

### P-Selectin Expression is Increased in JAK2V617F Endothelial Cells

The adhesion of leukocytes to endothelial cells is mediated by Cell Adhesion Molecules (CAM) and selectins²¹. Flow cytometry analysis showed that JAK2V617F HUVECs expressed inter-CAM (ICAM), vascular-CAM (VCAM) and E-selectin at the same levels than JAK2WT HUVECs, whether or not they were previously activated with TNF-alpha. Immunostaining of non permeabilized carotid arteries from Pdgfb-iCreERT2;JAK2^{V617F/WT} mice showed an increased exposure of P-selectin at the endothelial cell surface in vivo, whether or not they were previously administered with TNF-alpha. Cell-surface P-selectin is susceptible to proteolytic cleavage that results in the shedding of its extracellular domain in the circulation. We thus reasoned that soluble P-selectin might be increased in Pdgfb-iCreERT2;JAK2^{V617F/WT} mice. Using ELISA quantification, we observed increased levels of soluble P-selectin in the plasma of Pdgfb-iCreERT2;JAK2^{V617F/WT} mice, even after normalization with platelet count. Lastly we excluded increased platelet activation in Pdgfb-iCreERT2;JAK2^{V617F/WT} mice by quantifying soluble Platelet Factor 4. Altogether, these results are in favor of increased membrane-attached and plasmatic soluble P-selectin from endothelial origin without increase in endothelial ICAM, VCAM or E-selectin. Interestingly, the levels of soluble P-selectin and the number of rolling leukocytes correlated in both groups (Pdgfb-iCreERT2;JAK2^{V617F/WT} mice and control) (Pearson correlation test, r=0.8244), indicating that increased P-selectin expression is associated with increased leukocyte rolling.

### Endothelial Expression and Release of Von Willebrand Factor is Increased by JAK2V617F

Within endothelial cells, P-selectin is stored in exocytotic organelles called Weibel-Palade bodies together with von Willebrand factor (vWF). Exocytosis of Weibel Palade bodies leads to cell surface expression of vWF and P-selectin. In vitro, using immunostaining on non-permeabilized HUVECs, we observed increased vWF expression at the surface of JAK2V617F HUVECs, spontaneously and after overnight activation with 10 ng/ml TNF-alpha. Furthermore, quantification of vWF in the supernatant of HUVECs revealed higher amounts of vWF released by JAK2V617F HUVECs. Intra-cellular concentration of vWF was also increased in JAK2V617F HUVECs. Treating cells with TNF-alpha increased the secretion of vWF by JAK2WT HUVECs and to even greater levels by JAK2V617F HUVECs. Intracellular vWF levels were strongly reduced in TNF-treated cells and differences between JAK2V617F and JAK2WT HUVECs was abrogated after TNF-alpha treatment as the majority of Weibel-Palade bodies had likely already been released. These results were confirmed in vivo with higher levels of vWF antigen in Pdgfb-iCreERT2;JAK2^{V617F/WT} mice compared with control mice. All together, our data showed that endothelial JAK2V617F increased vWF protein level and soluble vWF release, in line with increased P-Selectin expression at cell surface as a consequence of increased degranulation of Weibel-Palade bodies.

### Increased P-Selectin Exposure is Involved in the Pro-Adhesive and Pro-Thrombotic Phenotype of JAK2V617F Endothelial Cells

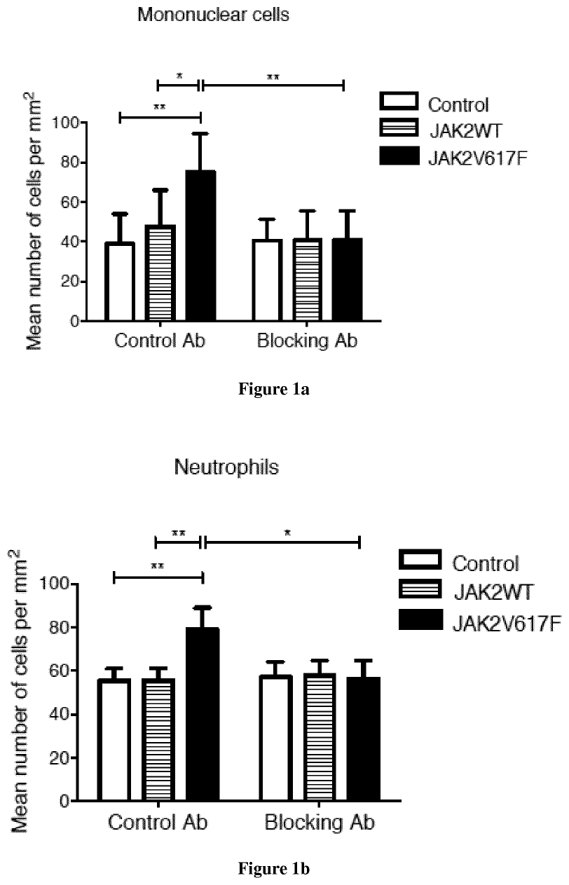

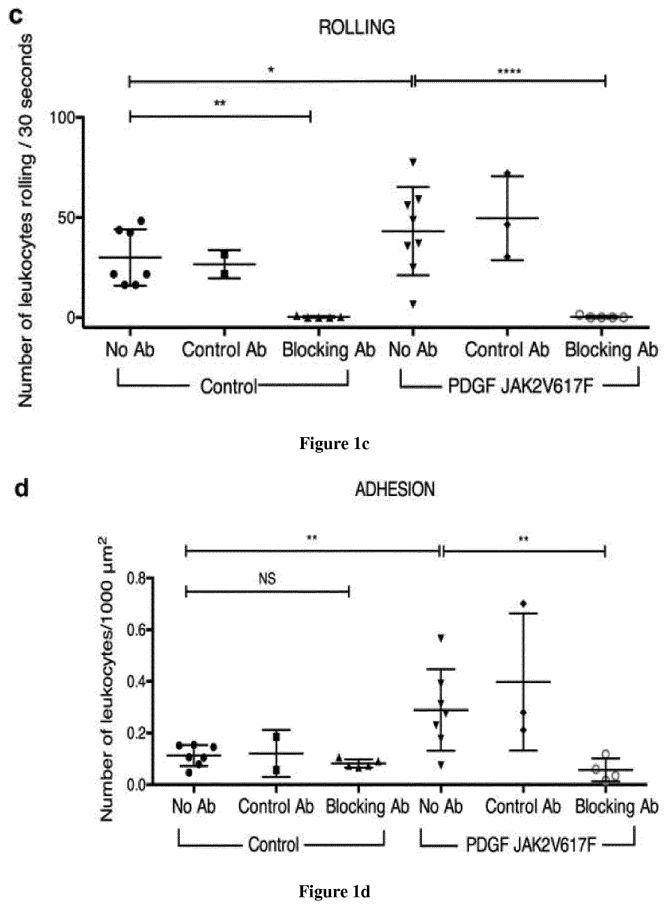

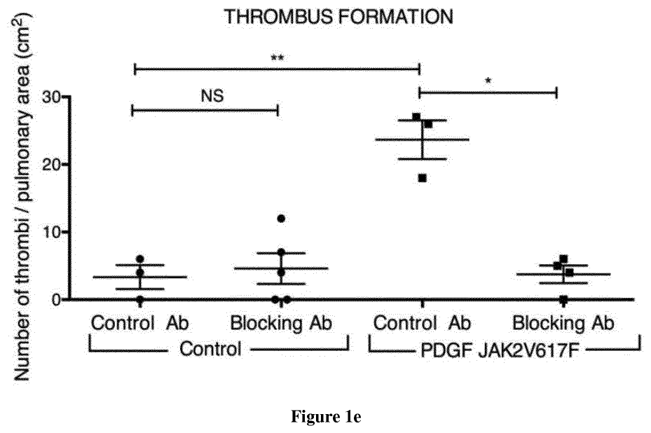

To investigate a potential causal link between increased P-Selectin exposure and the pro-adhesive phenotype of JAK2V617F endothelial cells, we reproduced the same experiments as previously described, but in the presence of a P-selectin blocking antibody. Our in vitro approach showed a complete reversion of the hyperadhesive properties of JAK2V617F HUVECs after exposition with the P-selectin blocking antibody (FIGS. 1a and b). Quantification of leukocytes in Pdgfb-iCreERT2;JAK2^{V617F/WT} mice treated with TNF-alpha and the P-selectin blocking antibody revealed a complete inhibition of leukocyte rolling and adhesion (FIG. 1c and d). To examine whether increased P-selectin was also responsible for thrombus formation, we used the model of low dose TNF-alpha induced lung thrombus formation. We observed that pre-treatment of the mice with the P-selectin blocking antibody completely abrogated thrombus formation in Pdgfb-iCreERT2;JAK2^{V617F/WT} mice, but had no effect in control mice (FIG. 1e). Altogether, these results show that the pro-thrombotic phenotype of JAK2V617F endothelial cells is mainly the consequence of increased adhesive properties, due to overexpression of membrane P-selectin, secondary to degranulation of Weibel-Palade bodies.

### Treatment with Hydroxyurea Abrogates TNF-Alpha Induced Thrombosis in Pdgfb-iCreERT2;JAK2^{V617F/WT} Mice Through Decreased P-Selectin Expression

Hydroxyurea is an antimetabolite frequently used in MPN to reduce the occurrence of thrombosis. Its anti-thrombotic effect is reported to be via the reduction of blood cell counts. But hydroxyurea is also used in sickle cell disease to reduce vasoocclusive crisis, and its beneficial effect is in part mediated by a direct effect on endothelial cells, with a reduction in leukocyte adhesion²². We wondered whether hydroxyurea was capable of reducing the prothrombotic effect of JAK2V617F endothelial cells. We treated Pdgfb-iCreERT2;JAK2^{V617F/WT} and control mice during 10 days with hydroxyurea, injected low dose TNF-alpha and quantified thrombus formation in lungs. We observed a significant inhibition of thrombus formation in Pdgfb-iCreERT2;JAK2^{V617F/WT} mice together with a reduction in leukocyte rolling and adhesion. In vitro, we observed a significant reduction of neutrophil adhesion on JAK2V617F HUVECs that had been pretreated 24 hours with hydroxyurea, demonstrating that the anti-adhesive effect of hydroxyurea observed in vivo is a direct effect on endothelial cells. We then examined whether the protective effect of hydroxyurea acted through reduction of endothelial P-selectin. We observed that P-selectin/platelets ratio was significantly lower in hydroxyurea-treated than in non-treated Pdgfb-iCreERT2;JAK2^{V617F/WT} mice. In these same mice, we observed a significant decrease of endothelial membrane P selectin expression, confirming a direct effect of hydroxyurea on JAK2V617F endothelial cells. Lastly, we measured vWF in the supernatant of JAK2V617F HUVECs that had been treated during 24 hours with 100 μM hydroxyurea. We observed decreased vWF concentrations, in favor of a reduced release of Weibel Palade bodies. Altogether, our in vitro and in vivo results show that hydroxyurea has a direct effect on JAK2V617F endothelial cells, decreasing endothelial P-selectin release and surface expression, thus decreasing endothelial cell pro-thrombotic phenotype.

### Discussion

Despite significant advances in deciphering the molecular mechanisms responsible for MPN occurrence and transformation, the mechanisms that lead to thrombosis, the first cause of morbidity and mortality, remain largely elusive. Recent identification of the JAK2V617F mutation in endothelial cells of MPN patients¹⁵⁻¹⁷ opens new perspectives in the pathogenesis of thrombosis in MPNs. Here we demonstrate using a transgenic mouse model, that JAK2V617F-positive endothelial cells promote spontaneous thrombosis in basal conditions and have increased thrombotic response to weak inflammatory stimuli. We demonstrate that the mechanism that leads to thrombosis involves endothelial P selectin release and cell surface exposure and subsequent leukocyte rolling and adhesion. We also demonstrate that treatment with hydroxyurea decreases P-selectin endothelial expression and thrombus formation in mice expressing JAK2V617F only in endothelial cells. The link between P-selectin expression and thrombosis has already been described, and in most cases P-selectin is from platelet origin²³⁻²⁵. The mechanism of P-selectin mediated thrombosis involves neutrophil activation, either through tissue factor expression and activation of the extrinsic coagulation pathway, or through priming for neutrophil extracellular trap formation²⁶, a process that is now well recognized as participating in thrombus formation²⁷. Increase of endothelial P-selectin and subsequent thrombosis was reported in response to venous flow reduction and local hypoxia²⁴. Here, we show for the first time that endothelial cells can have a constitutive increased expression of P-selectin, even without any hypoxic or inflammatory stimuli. Further studies are now required to decipher the specific molecular mechanism that is responsible for increased endothelial P-selectin expression in JAK2V617F expressing endothelial cells.

Given that MPN are acquired hematological malignancies, the description of prothrombotic JAK2V617F endothelial cells raises the question of their origin. JAK2V617F endothelial cells have been found using two approaches: culture of endothelial progenitors^{17,28-30} and microdissection^{15,16}. Endothelial progenitors comprise (i) Colony Forming Unit-Endothelial Cell (CFU-EC) which are of hematopoietic origin, give rise to endothelial cells unable to proliferate nor form vessels in transplantation experiments, and (ii) Endothelial Colony Forming Cells (ECFC) which generate a progeny of phenotypically and functionally competent endothelial cells. Three groups have looked for the presence of JAK2V617F in ECFC and CFU-EC in MPN patients. Two out of three found JAK2V617F ECFC^{17,30}, suggesting that the JAK2V617F mutation would have occurred in a progenitor cell of the hematopoietic and endothelial lineages. Such a cell certainly exists in the embryo but its existence in adults is a matter of debate. On the contrary, all groups found JAK2V617F CFU-EC in all MPN patients, a result that is not surprising given that CFU-EC are of hematopoietic origin. In the case of microdissection experiments^{15,16}, it is possible that the JAK2V617F endothelial cells that have been microdissected are of hematopoietic origin, as monocytes are known to integrate into the vessel wall after an injury, acquiring the phenotype of mature endothelial cells³¹. Altogether, in MPN, the presence of JAK2V617F endothelial cells of real endothelial origin is probably rare but the presence of JAK2V617F endothelial cells of hematopoietic origin is common. Such cells probably integrate into the vessel wall after a vascular lesion, thus giving rise to a bedding of JAK2V617F endothelial cells. Demonstrating that JAK2V617F endothelial cells have a prothrombotic phenotype is thus particularly relevant in our understanding of the pathogenesis of thrombosis in MPN.

Our study has important therapeutic implications. We demonstrate that treatment with hydroxyurea inhibits the pathological hyperadhesive phenotype of JAK2V617F endothelial cells. The results presented here challenge current thinking, according to which the antithrombotic effect of hydroxyurea in MPN is only mediated by lowering blood cell count. However, a direct effect of hydroxyurea on endothelial cells was already reported with increased NO and cGMP production³², probably via inhibition of eNOS degradation by proteasome³³. Moreover, in patients with sickle cell disease, treatment with hydroxyurea efficiently decreases vaso-occlusive crisis frequency³⁴. This antithrombotic effect was shown to be a direct effect on endothelial cells via stimulation of the NO-cGMP pathway, and reduction of leukocyte rolling in the microvasculature of TNF-alpha treated sickle cell mice²². Our result suggests that hydroxyurea should be considered in priority in patients with MPN and a history of thrombosis. This is often the case as hydroxyurea is the first line therapy in these high-risk patients. But some patients, due to young age or intolerance to high doses of hydroxyurea, take second line therapies such as interferon or anagrelide. We question whether they would still benefit from hydroxyurea treatment in association with other drugs to maintain thrombosis protection.

Another therapeutic implication of our work comes from our demonstration that increased endothelial P-selectin favors thrombosis in MPN. In sickle cell disease, various factors such as thrombin, platelet-activating factor, TNF-alpha, and sickle cells themselves are responsible for endothelial activation. Increased endothelial P-selectin expression was shown to participate in the occurrence of vasoocclusive crisis³⁵ and very recently, a clinical trial demonstrated that treatment with an anti-P-selectin antibody, crizanlizumab, efficiently prevented pain crises in sickle cell disease³⁶. It is thus tempting to speculate that such treatment could have therapeutical benefits in MPN patients with high thrombotic risk, who need to receive anticoagulant treatment for a MPN-related thrombosis (such as splanchnic thrombosis).

We believe our work raises a new concept as we show for the first time that endothelial cells bearing a genetic mutation acquire a prothrombotic phenotype. It is well known that endothelial cells, when stimulated by extrinsic factors such as inflammatory cytokines, hypoxia or antiphospholipid antibodies, become activated and promote thrombosis. There are some examples of constitutional mutations affecting endothelial cell functions and particularly angiogenic properties^{37,38} but, to our knowledge, no constitutional or acquired mutation affecting endothelial cell prothrombotic profile had been described to date. Using a mouse model with a specific expression of JAK2V617F in endothelial cells mimicking the presence of JAK2V617F in endothelial cells found in patients¹⁵⁻¹⁷, we provide insights into the pathogenesis of thrombosis in MPN, describe the pathological phenotype of intrinsically mutated endothelial cells, and provide evidences for the use of hydroxyurea in preventing thrombosis.

Altogether, we believe that our work is clinically relevant for three reasons: (i) it suggests that specific biomarkers of endothelial cell activation should be intensively looked for to determine which MPN patients are at high thrombotic risk. (ii) it opens the route to new therapeutic options in MPN, such as hydroxyurea in patients with high thrombotic risk or high endothelial activation markers, and anti-P-selectin antibody instead or in addition to standard care to prevent thrombosis in high risk MPN patients. (iii) it provides the proof of concept that an acquired genetic mutation can alter the phenotype of endothelial cells as shown for the acquired prothrombotic phenotype acquired upon expression of the JAK2 mutation. This suggests that other activating mutations in endothelial cells could be causal in thrombotic disorders of unknown cause, which account for 50% of recurrent venous thrombosis.

### REFERENCES

Throughout this application, various references describe the state of the art to which this invention pertains. The disclosures of these references are hereby incorporated by reference into the present disclosure.

1. Arber, D. A., et al. The 2016 revision to the World Health Organization classification of myeloid neoplasms and acute leukemia. Blood 127, 2391-2405 (2016).

2. Baxter, E. J., et al. Acquired mutation of the tyrosine kinase JAK2 in human myeloproliferative disorders. Lancet 365, 1054-1061 (2005).

3. James, C., et al. A unique clonal JAK2 mutation leading to constitutive signalling causes polycythaemia vera. Nature 434, 1144-1148 (2005).

4. Kralovics, R., et al. A gain-of-function mutation of JAK2 in myeloproliferative disorders. The New England journal of medicine 352, 1779-1790 (2005).

5. Levine, R. L., et al. Activating mutation in the tyrosine kinase JAK2 in polycythemia vera, essential thrombocythemia, and myeloid metaplasia with myelofibrosis. Cancer cell 7, 387-397 (2005).

6. Elliott, M. A. & Tefferi, A. Pathogenesis and management of bleeding in essential thrombocythemia and polycythemia vera. Current hematology reports 3, 344-351 (2004).

7. Barbui, T., Finazzi, G. & Falanga, A. Myeloproliferative neoplasms and thrombosis. Blood 122, 2176-2184 (2013).

8. Falanga, A., Marchetti, M., Vignoli, A., Balducci, D. & Barbui, T. Leukocyte-platelet interaction in patients with essential thrombocythemia and polycythemia vera. Experimental hematology 33, 523-530 (2005).

9. Barbui, T., Carobbio, A., Rambaldi, A. & Finazzi, G. Perspectives on thrombosis in essential thrombocythemia and polycythemia vera: is leukocytosis a causative factor? Blood 114, 759-763 (2009).

10. Landolfi, R., et al. Leukocytosis as a major thrombotic risk factor in patients with polycythemia vera. Blood 109, 2446-2452 (2007).

11. De Grandis, M., et al. JAK2V617F activates Lu/BCAM-mediated red cell adhesion in polycythemia vera through an EpoR-independent Rap1/Akt pathway. Blood 121, 658-665 (2013).

12. Alonci, A., et al. Evaluation of circulating endothelial cells, VEGF and VEGFR2 serum levels in patients with chronic myeloproliferative diseases. Hematological oncology 26, 235-239 (2008).

13. Belotti, A., et al. Circulating endothelial cells and endothelial activation in essential thrombocythemia: results from CD146+ immunomagnetic enrichment-flow cytometry and soluble E-selectin detection. American journal of hematology 87, 319-320 (2012).

14. Cella, G., et al. Nitric oxide derivatives and soluble plasma selectins in patients with myeloproliferative neoplasms. Thrombosis and haemostasis 104, 151-156 (2010).

15. Sozer, S., et al. The presence of JAK2V617F mutation in the liver endothelial cells of patients with Budd-Chiari syndrome. Blood 113, 5246-5249 (2009).

16. Rosti, V., et al. Spleen endothelial cells from patients with myelofibrosis harbor the JAK2V617F mutation. Blood 121, 360-368 (2012).

17. Teofili, L., et al. Endothelial progenitor cells are clonal and exhibit the JAK2(V617F) mutation in a subset of thrombotic patients with Ph-negative myeloproliferative neoplasms. Blood 117, 2700-2707 (2011).

18. Wakefield, T. W., Myers, D. D. & Henke, P. K. Mechanisms of venous thrombosis and resolution. Arteriosclerosis, thrombosis, and vascular biology 28, 387-391 (2008).

19. Hasan, S., et al. JAK2V617F expression in mice amplifies early hematopoietic cells and gives them a competitive advantage that is hampered by IFNalpha. Blood 122, 1464-1477 (2013).

20. Ollivier, V., et al. Bioreactivity of stent material: Activation of platelets, coagulation, leukocytes and endothelial cell dysfunction in vitro. Platelets, 1-11 (2016).

21. Ley, K., Laudanna, C., Cybulsky, M. I. & Nourshargh, S. Getting to the site of inflammation: the leukocyte adhesion cascade updated. Nature reviews 7, 678-689 (2007).

22. Almeida, C. B., et al. Hydroxyurea and a cGMP-amplifying agent have immediate benefits on acute vaso-occlusive events in sickle cell disease mice. Blood 120, 2879-2888 (2012).

23. Andre, P., Hartwell, D., Hrachovinova, I., Saffaripour, S. & Wagner, D. D. Pro-coagulant state resulting from high levels of soluble P-selectin in blood. Proceedings of the National Academy of Sciences of the United States of America 97, 13835-13840 (2000).

24. von Bruhl, M. L., et al. Monocytes, neutrophils, and platelets cooperate to initiate and propagate venous thrombosis in mice in vivo. The Journal of experimental medicine 209, 819-835 (2012).

25. Zetterberg, E., et al. Abnormal P-selectin localization during megakaryocyte development determines thrombosis in the gata1low model of myelofibrosis. Platelets 25, 539-547 (2014).

26. Etulain, J., et al. P-selectin promotes neutrophil extracellular trap formation in mice. Blood 126, 242-246 (2015).

27. Martinod, K. & Wagner, D. D. Thrombosis: tangled up in NETs. Blood 123, 2768-2776 (2014).

28. Helman, R., et al. Granulocyte whole exome sequencing and endothelial JAK2V617F in patients with JAK2V617F positive Budd-Chiari Syndrome without myeloproliferative neoplasm. British journal of haematology (2016).

29. Piaggio, G., et al. Endothelial colony-forming cells from patients with chronic myeloproliferative disorders lack the disease-specific molecular clonality marker. Blood 114, 3127-3130 (2009).

30. Yoder, M. C., et al. Redefining endothelial progenitor cells via clonal analysis and hematopoietic stem/progenitor cell principals. Blood 109, 1801-1809 (2007).

31. Murdoch, C., Muthana, M., Coffelt, S. B. & Lewis, C. E. The role of myeloid cells in the promotion of tumour angiogenesis. Nature reviews. Cancer 8, 618-631 (2008).

32. Cokic, V. P., et al. Hydroxyurea induces the eNOS-cGMP pathway in endothelial cells. Blood 108, 184-191 (2006).

33. Cokic, V. P., Beleslin-Cokic, B. B., Noguchi, C. T. & Schechter, A. N. Hydroxyurea increases eNOS protein levels through inhibition of proteasome activity. Nitric oxide: biology and chemistry 16, 371-378 (2007).

34. Charache, S., et al. Effect of hydroxyurea on the frequency of painful crises in sickle cell anemia. Investigators of the Multicenter Study of Hydroxyurea in Sickle Cell Anemia. The New England journal of medicine 332, 1317-1322 (1995).

35. Matsui, N. M., et al. P-selectin mediates the adhesion of sickle erythrocytes to the endothelium. Blood 98, 1955-1962 (2001).

36. Ataga, K. I., et al. Crizanlizumab for the Prevention of Pain Crises in Sickle Cell Disease. The New England journal of medicine 376, 429-439 (2017).

37. Labauge, P., Denier, C., Bergametti, F. & Tournier-Lasserve, E. Genetics of cavernous angiomas. The Lancet. Neurology 6, 237-244 (2007).

38. Larrivee, B., et al. ALK1 signaling inhibits angiogenesis by cooperating with the Notch pathway. Developmental cell 22, 489-500 (2012).

39. Falati, S., et al. Accumulation of tissue factor into developing thrombi in vivo is dependent upon microparticle P-selectin glycoprotein ligand 1 and platelet P-selectin. The Journal of experimental medicine 197, 1585-1598 (2003).

40. Su, Y., Lei, X., Wu, L. & Liu, L. The role of endothelial cell adhesion molecules P-selectin, E-selectin and intercellular adhesion molecule-1 in leucocyte recruitment induced by exogenous methylglyoxal. Immunology 137, 65-79 (2012).

41. Dargaud, Y., et al. Evaluation of a standardized protocol for thrombin generation measurement using the calibrated automated thrombogram: an international multicentre study. Thrombosis research 130, 929-934 (2012).

42. Claxton, S., et al. Efficient, inducible Cre-recombinase activation in vascular endothelium. Genesis 46, 74-80 (2008).

43. Chauhan, A. K., et al. ADAMTS13: a new link between thrombosis and inflammation. The Journal of experimental medicine 205, 2065-2074 (2008).

44. Wegmann, F., et al. ESAM supports neutrophil extravasation, activation of Rho, and VEGF-induced vascular permeability. The Journal of experimental medicine 203, 1671-1677 (2006).

45. Rayes, J., et al. Mutation and ADAMTS13-dependent modulation of disease severity in a mouse model for von Willebrand disease type 2B. Blood 115, 4870-4877 (2010).

1. A method of treating or prophylactically treating thrombosis in a patient suffering from a myeloproliferative neoplasm comprising administering to the patient a therapeutically effective amount of a P-selectin antagonist.

2. The method of claim 1 wherein the patient suffers from polycythemia vera (PV), essential thrombocythemia (ET) or primary myelofibrosis (PMF).

3. The method of claim 1 wherein the patient harbours one mutation in JAK2.

4. The method of claim 3 wherein the one mutation is the JAK2V617F mutation.

5. The method of claim 1 wherein the P-selectin antagonist is administered to the patient for prophylactically treating thrombosis.

6. The method of claim 1 wherein the P-selectin antagonist is an antibody against P-selectin.

7. The method of claim 6 wherein the antibody is Crizanlizumab.

8. The method of claim 1 wherein the P-selectin antagonist is hydroxycarbamide.

9. The method of claim 1 wherein the P-selectin antagonist is an inhibitor of P-selectin expression.

10. The method of claim 10, wherein the inhibitor of P-selectin expression is a siRNA or an antisense oligonucleotide.
